(19) United States
(12) Patent Application Publication
(10) Pub. No.: US 2026/0306413 A1
(43) Pub. Date: Oct. 8, 2026

(54) COLONIC PURGATIVE TABLET AND COLONIC PURGATIVE COMPRISING SAME

(71) Applicant: PHARMBIO KOREA CO., LTD., Chungcheongbuk-do (KR)

(72) Inventors: Bong Kil NAM, Seoul (KR); Jun Sang NAM, Seoul (KR); Hyun Jung JUNG, Seoul (KR); Hyun Il KIM, Gyeonggi-do (KR); Kyung Su LEE, Gyeonggi-do (KR)

(73) Assignee: PHARMBIO KOREA CO., LTD., Chungcheongbuk-do (KR)

(21) Appl. No.: 19/483,328

(22) PCT Filed: May 10, 2024
(86) PCT No.: PCT/KR2024/006414

§ 371 Date: Nov. 11, 2025

(30) Foreign Application Priority Data
May 12, 2023 (KR) 1020230061890

(51) Int. Cl. A61K 9/28; A61K 9/00; A61K 9/20

(52) U.S. Cl. CPC A61K 9/286; A61K 9/0004; A61K 9/2009; A61K 9/2027; A61K 9/2036; A61K 9/2095; A61K 9/2853; A61K 9/2893

(57) ABSTRACT

The present invention can provide a colonic purgative tablet and a colonic purgative comprising same, the tablet having a gel-forming coating layer, which contains alginate as a main component, introduced into an uncoated tablet containing an osmotic material, so that, while damage to the gastric mucosa due to physical collision between the tablet and the gastric mucosa is be prevented, the osmotic material contained in the uncoated tablet rapidly disintegrates, and thus an excellent bowel cleansing effect is provided. The colonic purgative tablet according to the present invention can exhibit an excellent bowel cleansing effect even in small doses, and can further improve medication compliance through selective miniaturization of the tablet or introduction of an adhesive improvement coating layer.

### TECHNICAL FIELD

The present disclosure relates to a colonic purgative tablet and a colonic purgative comprising the same, and more specifically, to a colonic purgative tablet comprising a gel-forming coating layer comprising alginate as a main ingredient, preventing damage to the gastric mucosa injury and having an excellent intestine cleansing effect, and a colonic purgative comprising the same.

### BACKGROUND ART

To increase the efficiency of colonoscopy, colon cleansing is required. Colon cleansing is achieved by adjusting diet and taking a colonic purgative before the test.

Drugs used as colonic purgatives are classified into osmotic laxatives, stimulant laxatives, and saline laxatives depending on their mechanism of action. Osmotic laxatives are non-absorbable electrolyte solutions that, after taking, create an osmotic pressure difference in the intestines to wash away feces. Stimulant laxatives interfere with the absorption of water and electrolytes in the colon and stimulate the intestinal mucosa, inducing contraction of the colon muscles and forcing defecation. Saline laxatives are not absorbed in the intestinal tract and retain water from the small intestine through osmotic pressure, thereby diluting stool and at the same time activating peristalsis to induce defecation.

An example of an osmotic laxative is polyethylene glycol (PEG) solution. PEG has little effect on plasma volume and electrolytes, so it is suitable for patients with concomitant diseases such as kidney disease, heart disease, and liver disease. However, it requires taking a lot of solution (4 liters) and has a very unpleasant taste, so medication compliance is low. There is a product that is a combination of PEG and ascorbic acid and shows a cleansing effect with only 2 liters, but the dosage is still burdensome due to the unique unpleasant taste of PEG.

Examples of irritant laxatives comprise a combination of sodium picosulfate hydrate, magnesium oxide, and citric acid. For adults, 170 ml of solution is taken before 8 a.m. the day before the test, and an additional 170 ml of solution is taken 6-8 hours later. It has the advantage of significantly reducing the dosage compared to PEG preparations. However, it is recommended to drink 250 ml of water per hour after taking it to avoid dehydration, so medication compliance is still not considered excellent.

Examples of saline laxatives comprise phosphates and sulfates. Phosphate has been reported to have a colon cleansing effect in smaller doses compared to PEG, but it causes side effects such as abdominal inflation, pain, and dizziness at a similar frequency to PEG, and the European Society of Gastroenterology restricted the use of oral sodium phosphate in 2013 because it was found to cause complications, particularly acute phosphate nephropathy. On the other hand, sulfate is evaluated as being relatively safer than phosphate. As sulfate preparations, a combination of sodium sulfate, potassium sulfate, and sodium sulfate is mainly used.

Korean Patent Publication No. 10-2015-0089430 discloses a colonic purgative tablet prepared by adding and mixing PEG as an excipient rather than the main ingredient to a mixture of magnesium sulfate, potassium sulfate, and sodium sulfate, then adding sodium stearyl fumarate, and compressing it in a tablet press. The document introduces that a product consisting of three types of sulfates, magnesium sulfate, potassium sulfate, and sodium sulfate, was reported to be safe and effective, and a product called the American Suprep Bowel Prep Kit was also developed. Products consisting of the above three sulfates comprise Suprep and Suclear solutions in Korea. However, liquid preparations have the matters of poor medication compliance due to the salty taste and foul smell unique to sulfate, and excessive sweeteners and flavoring agents are sometimes added to eliminate the unique rotten egg smell of sulfate, but the effect is limited.

In order to solve the matters of colonic purgatives in the related art as described above, the present applicant has proposed a colonic purgative in the form of a solid preparation comprising the main ingredients consisting of anhydrous magnesium sulfate, potassium sulfate, anhydrous sodium sulfate, and simethicone through Korean Patent Publication No. 10-2019-0041233, and the colonic purgative according to the relevant literature is marketed under the name Orafang® tab. Orafang® tab has various advantages, such as improving medication compliance and having an excellent intestine cleansing effect. However, after the marketing of Orafang® tab, some patients reported gastric mucosa injury and discomfort when swallowing the tablet due to its large size. Accordingly, there is a need to develop a colonic purgative that can solve these matters and have an excellent intestine cleansing effect.

### DISCLOSURE OF INVENTION

### Technical Problem

The present disclosure is for the purpose of providing a colonic purgative tablet in which the osmotic substance comprised in the uncoated tablet is rapidly disintegrated and may have an excellent intestine cleansing effect even at a small dosage while preventing injury to the gastric mucosa due to physical collision between the tablet and the gastric mucosa, by introducing a gel-forming coating layer comprising alginate as a main ingredient into an uncoated tablet comprising an osmotic substance.

The present disclosure is also for the purpose of providing a colonic purgative tablet that may be easily taken through a packaging bag.

### Solution to Problem

The present disclosure relates to a colonic purgative tablet comprising: an uncoated tablet comprising an osmotic substance, simethicone, and a water-soluble binder; and a gel-forming coating layer comprising alginate as a main ingredient formed on the uncoated tablet. The osmotic substance comprises one or more selected from the group consisting of anhydrous sodium sulfate, potassium sulfate, anhydrous magnesium sulfate, sodium sulfate hydrate, magnesium sulfate hydrate, sodium picosulfate hydrate, light magnesium oxide, citric acid, polyethylene glycol, sodium chloride, potassium chloride, ascorbic acid, sodium ascorbate, sodium hydrogen carbonate, sodium hydrogen phosphate anhydride, sodium dihydrogen phosphate monohydrate, magnesium oxide, and potassium sulfate, and the alginate is comprised in an amount of 50% by weight or more based on the total weight of the gel-forming coating layer.

In one embodiment, the osmotic substance may comprise anhydrous sodium sulfate, potassium sulfate, and anhydrous magnesium sulfate.

In one embodiment, the colonic purgative tablet may comprise 60 to 90 parts by weight of anhydrous sodium sulfate, 5 to 25 parts by weight of potassium sulfate, and 1 to 15 parts by weight of anhydrous magnesium sulfate, based on 100 parts by weight of the osmotic substance.

In one embodiment, the colonic purgative tablet may comprise 0.1 to 2 parts by weight of simethicone and 0.5 to 20 parts by weight of a water-soluble binder based on 100 parts by weight of the osmotic substance.

In one embodiment, the water-soluble binder may be one or more selected from the group consisting of copovidone, polyethylene glycol, and povidone.

In one embodiment, the alginate may be one or more selected from the group consisting of sodium alginate, potassium alginate, and magnesium alginate.

In one embodiment, the gel-forming coating layer may further comprise an adhesion improving agent.

In one embodiment, the adhesion improving agent is selected from the group consisting of polyvinyl alcohol-polyethylene glycol graft copolymer, hypromellose, hydroxypropylcellulose, hydroxyethylcellulose, pol, gelatin, carboxymethylcellulose, and povidone. There may be more than one selected.

In one embodiment, the gel-forming coating layer may further comprise a mucosa protective agent.

In one embodiment, the mucosa protective agent may be one or more selected from the group consisting of ecabet sodium hydrate, isogladine maleate, and polaprezinc.

In one embodiment, the colonic purgative tablet may further comprise the adhesion-improving coating layer comprising the adhesion improving agent as a main ingredient on the gel-forming coating layer.

In one embodiment, the colonic purgative tablet may comprise 50% by weight or more of the adhesion improving agent based on the total weight of the adhesion-improving coating layer.

In one embodiment, when evaluating the elution pattern of the osmotic substance in a solution of pH 2 or lower according to the second elution test method, the osmotic substance may be eluted at 30% by weight or less within 5 minutes.

In one embodiment, when evaluating the elution pattern of the osmotic substance in a solution of pH 3.5 to 5.5 according to the second elution test method, the osmotic substance may be eluted at 30% by weight or more within 5 minutes.

In one embodiment, when evaluating the elution pattern of the osmotic substance in a solution of pH 6 to 8.5 or lower according to the second elution test method, the osmotic substance may be eluted at 40% by weight or more within 5 minutes.

In addition, the present disclosure may provide a colonic purgative comprising two or more packaging bags comprising 2 or more tablets of the colonic purgative tablet, and taking the colonic purgative tablet comprised in one packaging bag for a single time.

In addition, the present disclosure may relate to a pharmaceutical composition for the colonic purgative. On the day before the colonoscopy, 160 tablets of the colonic purgative tablet of claim 1 and 425 ml of water are taken, then 850 ml of water is taken in two additional doses of 425 ml each, and on the day of the colonoscopy, colonic purgative tablets and water are repeatedly taken in the same manner as above, and the total dose of the colonic purgative is 38 to 55 g.

In addition, the present disclosure may relate to a pharmaceutical composition for colonic purgative. On the day of the colonoscopy, 160 tablets of the colonic purgative tablet and 425 ml of water are taken, then 850 ml of water is taken in two additional doses of 425 ml each, and the total dose of the colonic purgative is 38 to 55 g.

### Advantageous Effects of Invention

The present disclosure can provide a colonic purgative tablet in which the osmotic substance comprised in the uncoated tablet is rapidly disintegrated and may have an excellent intestine cleansing effect while preventing injury to the gastric mucosa due to physical collision between the tablet and the gastric mucosa, by introducing a gel-forming coating layer comprising alginate as a main ingredient into an uncoated tablet comprising an osmotic substance. The colonic purgative tablet according to the present disclosure may exhibit an excellent intestine cleansing effect even at a small dosage, and medication compliance may be further improved by selectively miniaturizing the tablet or introducing an adhesion-improving coating layer.

The present disclosure can also provide a colonic purgative tablet that may be easily taken through a packaging bag.

The effect of the present disclosure is not limited to the above-described effects, and it should be understood to comprise all effects that may be inferred from the configurations described in the description or claims of the present disclosure.

### BRIEF DESCRIPTION OF DRAWINGS

The above and other objects, features and advantages of the present disclosure will become more apparent to those of ordinary skill in the art by describing exemplary embodiments thereof in detail with reference to the accompanying drawings, in which:

FIG. 1 is an image taken of the tablet of Example 1 gelled in a solution with a pH of about 1.2;

FIG. 2 is an image showing the results of evaluating the gelation pattern according to pH for the tablet of Example 1;

FIG. 3 is an image showing the results of evaluating the dissolution pattern over time for the tablets of Examples and Comparative Examples;

FIG. 4 is a graph showing the results of evaluating the elution pattern of anhydrous sodium sulfate for the tablets of Examples and Comparative Examples; and

FIG. 5 is a schematic diagram showing the participation and processing status of clinical trial subjects.

### BEST MODE(S) FOR CARRYING OUT THE INVENTION

Hereinafter, the intention, operation, and effect of the present disclosure will be described in detail through embodiments of the present disclosure and specific examples thereof. However, the following Examples are presented as examples to aid understanding of the present disclosure as described above, and the scope of the invention is not limited or limited thereto.

At this time, the terms or words used in the specification and claims should not be interpreted as being limited to meanings in the related art or dictionary meanings, and the terms or words should be interpreted as a meaning and a concept that are consistent with the technical concept of the present disclosure based on the principle that the inventor can appropriately define the concepts of terms in order to explain his/her own invention in the best way.

Accordingly, it should be understood that the configuration of the embodiments described herein are only preferred embodiments of the present disclosure and are not intended to vary all of the spirit of the invention, and that there may be various equivalents and modifications that may be substituted for them at the time of this application.

In this specification, the singular expression comprises the plural expression unless the context clearly dictates otherwise.

Throughout the specification, when a part is said to “comprise” a component, this means that the part may further comprise other components rather than excluding other components unless specifically stated to the contrary. Thus, for example, a composition comprising compound A may comprise compounds other than A. However, the term “comprising” also, as a specific embodiment thereof, also encompasses the more limited meanings “consisting essentially/necessarily of” and “consisting of,” for example, “a composition comprising compound A” may also (essentially/necessarily) consist of compound A.

In connection with this, as used herein, the terms “comprises,” “comprises,” or “has” and the like are intended to specify the presence of the features, numbers, steps, components, or combinations thereof that are implemented, and are not to be understood as precluding the possibility of the presence or addition of one or more other features or numbers, steps, components or combinations thereof.

In this specification, when an arbitrary member is said to be located “on” another arbitrary member, this comprises not only the case where such member is in contact with another member, but also the case where another member or material are present between the two members.

In this specification, when an amount, concentration, or other value or parameter is given as either a range, preferred range, or a list of upper preferable values and lower preferable values, it is to be understood as specifically disclosing all ranges which may be formed from any pair of any upper range limit or preferred value and any lower range limitation or preferred values, regardless of whether ranges are separately disclosed. Where a range of numerical values is recited herein, unless otherwise stated, for example, the range is intended to comprise the endpoints thereof, and all integers and fractions within the range, unless the context clearly dictates otherwise. It is intended that the scope of the invention not be limited to the particular values recited when defining the ranges.

The present disclosure may relate to a colonic purgative tablet comprising: an uncoated tablet comprising an osmotic substance, simethicone, and a water-soluble binder; and/or a gel-forming coating layer comprising alginate as a main ingredient formed on the uncoated tablet. The osmotic substance may comprise one or more selected from the group consisting of anhydrous sodium sulfate, potassium sulfate, anhydrous magnesium sulfate, sodium sulfate hydrate, magnesium sulfate hydrate, sodium picosulfate hydrate, light magnesium oxide, citric acid, polyethylene glycol, sodium chloride, potassium chloride, ascorbic acid, sodium ascorbate, sodium hydrogen carbonate, sodium hydrogen phosphate anhydride, sodium dihydrogen phosphate monohydrate, magnesium oxide, and potassium sulfate, and the alginate may be comprised in an amount of 50% by weight or more based on the total weight of the gel-forming coating layer.

In one embodiment, the gel-forming coating layer comprising alginate as a main ingredient may comprise alginate in an amount of 60% by weight or more, 70% by weight or more, 80% by weight or more, 90% by weight or more, 95% by weight or more, 96% by weight or more, 97% by weight or more, 98% by weight or more, or 99% by weight or more, based on the total weight of the gel-forming coating layer.

In one embodiment, the alginate may be comprised in an amount of 0.1 to 20 parts by weight based on 100 parts by weight of the osmotic substance. In another embodiment, the alginate may be comprised in an amount of 0.2 parts by weight or more, 0.3 parts by weight or more, 0.4 parts by weight or more, 0.5 parts by weight or more, 0.6 parts by weight or more, 0.7 parts by weight or more, 0.8 parts by weight or more, 0.9 parts by weight or more, 1.0 parts by weight or more, 1.1 parts by weight or more, 1.2 parts by weight or more, 1.3 parts by weight or more, 1.4 parts by weight or more, 1.5 parts by weight or more, 2 parts by weight or more, 2.5 parts by weight or more, 3 parts by weight or more, 3.5 parts by weight or more, 4 parts by weight or more, or 4.5 parts by weight or more, and 18 parts by weight or less, 16 parts by weight or less, 14 parts by weight or less, 12 parts by weight or less, 10 parts by weight or less, 8 parts by weight or less, or 6 parts by weight or less, based on 100 parts by weight of the osmotic substance. As the colonic purgative tablet of the present disclosure contains alginate as described above, the gel-forming coating layer may be formed homogeneously with an appropriate thickness on the uncoated tablet.

In one embodiment, the thickness of the gel-forming coating layer may be within a range of 0.001 mm to 1 mm. In another embodiment, the thickness of the gel-forming coating layer may be 0.005 mm or more, 0.01 mm or more, 0.02 mm or more, 0.03 mm or more, 0.04 mm or more, 0.05 mm or more, 0.06 mm or more, 0.07 mm or more, 0.08 mm or more, or 0.09 mm or more, and 0.9 mm or less, 0.8 mm or less, 0.7 mm or less, 0.6 mm or less, 0.5 mm or less, 0.4 mm or less, 0.3 mm or less, or 0.2 mm or less.

The colonic purgative tablet of the present disclosure may prevent injury to the gastric mucosa due to physical collision between the tablet and the gastric mucosa while having an excellent intestine cleansing effect, by introducing a gel-forming coating layer on the uncoated tablet as described above.

In one embodiment, the osmotic substance may comprise anhydrous sodium sulfate, potassium sulfate, and anhydrous magnesium sulfate.

In one embodiment, the colonic purgative tablet may comprise 60 to 90 parts by weight of anhydrous sodium sulfate, 5 to 25 parts by weight of potassium sulfate, and 1 to 15 parts by weight of anhydrous magnesium sulfate, based on 100 parts by weight of the osmotic substance. In another embodiment, the anhydrous sodium sulfate may be comprised in an amount of 65 parts by weight or more, 70 parts by weight or more, or 75 parts by weight or more, and 85 parts by weight or less, or 80 parts by weight or less, based on 100 parts by weight of the osmotic substance In another embodiment, the potassium sulfate may be comprised in an amount of 7 parts by weight or more, 9 parts by weight or more, 11 parts by weight or more, or 13 parts by weight or more, and 23 parts by weight or less, 21 parts by weight or less, 19 parts by weight or less, 17 parts by weight or less, or 15 parts by weight or less, based on 100 parts by weight of the osmotic substance. In another embodiment, the anhydrous magnesium sulfate may be comprised in an amount of 2 parts by weight or more, 3 parts by weight or more, 4 parts by weight or more, 5 parts by weight or more, or 6 parts by weight or more, and 14 parts by weight or less, 13 parts by weight or less, 12 parts by weight or less, 11 parts by weight or less, 10 parts by weight or less, 9 parts by weight or less, or 8 parts by weight or less, based on 100 parts by weight of the osmotic substance.

The colon cleansing tablet of the present disclosure may have an excellent colon cleansing effect even at a small dosage by controlling the type and/or weight ratio of the osmotic substance as described above, thereby improving medication compliance, and this effect may be further maximized by introducing simethicone and/or a water-soluble binder as follows.

In one embodiment, the colonic purgative tablet of the present disclosure may comprise 0.1 to 2 parts by weight of simethicone and/or 0.5 to 20 parts by weight of a water-soluble binder, based on 100 parts by weight of the osmotic substance. In another embodiment, the colonic purgative tablet of the present disclosure may comprise 0.2 parts by weight or more, 0.3 parts by weight or more, 0.4 parts by weight or more, 0.5 parts by weight or more, 0.6 parts by weight or more, or 0.7 parts by weight or more, and 1.9 parts by weight or less, 1.8 parts by weight or less, 1.7 parts by weight or less, 1.6 parts by weight or less, 1.5 parts by weight or less, 1.4 parts by weight or less, 1.3 parts by weight or less, 1.2 parts by weight or less, 1.1 parts by weight or less, 1.0 parts by weight or less, or 0.9 parts by weight or less of simethicone, based on 100 parts by weight of the osmotic substance. In another embodiment, the colonic purgative tablet of the present disclosure may comprise 1 parts by weight or more, 1.5 parts by weight or more, or 2 parts by weight or more, and 18 parts by weight or less, 16 parts by weight or less, 14 parts by weight or less, or 12 parts by weight or less of the water-soluble binder, based on 100 parts by weight of the osmotic substance.

In one embodiment, the water-soluble binder may be one or more selected from the group consisting of copovidone, polyethylene glycol, and povidone, and from the viewpoint of improving the disintegration rate, copovidone or polyethylene glycol may be preferable, and polyethylene glycol may be more preferable.

In one embodiment, the polyethylene glycol may have a weight average molecular weight in a range of 3000 to 10000 g/mol. In another embodiment, the polyethylene glycol may have a weight average molecular weight of 4000 g/mol or more, or 5000 g/mol or more, and 9000 g/mol or less, 8000 g/mol or less, or 7000 g/mol or less. By introducing the polyethylene glycol with a weight average molecular weight within the above range, the disintegration rate may be further improved.

In one embodiment, the alginate may be one or more selected from the group consisting of sodium alginate, potassium alginate, and magnesium alginate, and it may be preferable that this is sodium alginate from the viewpoint of gelling in an intragastric environment without impeding the permeability of the tablet to prevent injury to the gastric mucosa and increase the disintegration rate improvement effect. As used herein, “intragastric environment” may mean the intragastric environment in a natural state and/or the intragastric environment in a state in which a large amount of water is input.

In one embodiment, the gel-forming coating layer may further comprise an adhesion improving agent.

In one embodiment, the adhesion improving agent may be one or more selected from the group consisting of a polyvinyl alcohol-polyethylene glycol graft copolymer, hypromellose, hydroxypropyl cellulose, hydroxyethyl cellulose, pol, gelatin, carboxymethyl cellulose, and povidone.

In one embodiment, the gel-forming coating layer may further comprise a mucosa protective agent.

In one embodiment, the mucosa protective agent may be one or more selected from the group consisting of an ecabet sodium hydrate, isogladine maleate, and polaprezinc.

The colonic purgative tablet of the present disclosure may further improve a gastric mucosa protective effect by selectively comprising the mucosa protective agent in the gel-forming coating layer.

In one embodiment, the colonic purgative tablet of the present disclosure may further comprise the adhesion-improving coating layer comprising the adhesion improving agent as a main ingredient on the gel-forming coating layer.

In one embodiment, the adhesion improving agent may be comprised in an amount of 50% by weight or more based on the total weight of the adhesion-improving coating layer. In another embodiment, the adhesion improving agent may be comprised in an amount of 60% by weight or more, 70% by weight or more, 80% by weight or more, or 90% by weight or more, based on the total weight of the adhesion-improving coating layer.

In one embodiment, the adhesion improving agent may be comprised in an amount of 0.1 to 20 parts by weight based on 100 parts by weight of the osmotic substance. In another embodiment, the adhesion improving agent may be comprised in an amount of 0.2 parts by weight or more, 0.3 parts by weight or more, 0.4 parts by weight or more, 0.5 parts by weight or more, 0.6 parts by weight or more, 0.7 parts by weight or more, 0.8 parts by weight or more, 0.9 parts by weight or more, 1.0 parts by weight or more, 1.1 parts by weight or more, 1.2 parts by weight or more, 1.3 parts by weight or more, 1.4 parts by weight or more, 1.5 parts by weight or more, 1.6 parts by weight or more, 1.7 parts by weight or more, and 18 parts by weight or less, 16 parts by weight or less, 14 parts by weight or less, 12 parts by weight or less, 10 parts by weight or less, 8 parts by weight or less, 6 parts by weight or less, or 4 parts by weight or less, based on 100 parts by weight of the osmotic substance. As an adhesion-improving coating layer is additionally comprised as described above, the colonic purgative tablet of the present disclosure can mask unpleasant taste or odor when ingesting the tablet and improve medication convenience by improving sticking in the mouth, and this effect can be further maximized when the gel-forming coating layer does not contain an adhesion improving agent.

In one embodiment, when evaluating the elution pattern of the osmotic substance in a solution of pH 2 or lower according to the second elution test method, the osmotic substance may be eluted at 30% by weight or less within 5 minutes. In the present specification, the osmotic substance being eluted by ‘a’ weight % may mean that the osmotic substance is eluted by ‘a’ weight % of the total weight of the osmotic substance comprised in the tablet. In another embodiment, when evaluating the elution pattern of the osmotic substance in a tablet in a solution of pH 2 or lower according to the elution test method 2, the osmotic substance may be eluted in an amount of 29% by weight or less, 28% by weight or less, 27% by weight or less, 26% by weight or less, 25% by weight or less, 24% by weight or less, 23% by weight or less, 22% by weight or less, 21% by weight or less, 20% by weight or less, 19.5% by weight or less, 19% by weight or less, or 18.5% by weight or less, and 1% by weight or more, 2% by weight or more, 3% by weight or more, 4% by weight or more, or 5% by weight or more within 5 minutes. In the present specification, when evaluating the elution pattern of the osmotic substance according to the elution test method 2, the osmotic substance may be, for example, anhydrous sodium sulfate.

In one embodiment, when evaluating the elution pattern of the osmotic substance in a solution of pH 3.5 to 5.5 according to the second elution test method, the osmotic substance may be eluted at 30% by weight or more within 5 minutes. In another embodiment, when evaluating the elution pattern of the osmotic substance in a tablet in a solution of pH 3.5 to 5.5 according to the elution test method 2, the osmotic substance may be eluted in an amount of 35% by weight or more, 40% by weight or more, 45% by weight or more, 50% by weight or more, 55% by weight or more, 60% by weight or more, 65% by weight or more, 70% by weight or more, 75% by weight or more, or 80% by weight or more, and 95% by weight or less, 90% by weight or less, 85% by weight or less, 80% by weight or less, 75% by weight or less, 70% by weight or less, 65% by weight or less, 60% by weight or less, or 55% by weight or less within 5 minutes.

In one embodiment, when evaluating the elution pattern of the osmotic substance in a solution of pH 6 to 8.5 or lower according to the second elution test method, the osmotic substance may be eluted at 40% by weight or more within 5 minutes. In another embodiment, when evaluating the elution pattern of the osmotic substance in a tablet in a solution of pH 6 to 8.5 according to the elution test method 2, the osmotic substance may be eluted in an amount of 45% by weight or more, 50% by weight or more, 55% by weight or more, 60% by weight or more, 65% by weight or more, 70% by weight or more, 75% by weight or more, 80% by weight or more, 85% by weight or more, or 90% by weight or more, and 99% by weight or less, 98% by weight or less, 95% by weight or less, 90% by weight or less, 85% by weight or less, or 80% by weight or less within 5 minutes.

In one embodiment, the colonic purgative tablet of the present disclosure may be round or rectangular, and from the viewpoint of medication convenience, it may be preferably circular with a diameter of 10 mm or less, more preferably 7 mm or less.

In addition, the present disclosure may relate to a colon laxative comprising 2 or more packaging bags comprising 2 or more tablets of the colonic purgative tablet, and taking the colonic purgative tablet comprised in one packaging bag for a single time.

In one embodiment, the total dose of the colonic purgative may be 38 to 55 g. As used herein, the total dose of the colonic purgative may refer to the total amount of the colonic purgative taken the day before and/or on the day of colonoscopy. In other embodiments, the total dose of the colonic purgative may be 39 g or more, 40 g or more, 41 g or more, or 42 g or more, and 54 g or less, 53 g or less, 52 g or less, 51 g or less, 50 g or less, 49 g or less, or 48 g or less.

In one embodiment, the colonic purgative tablet per packaging bag may comprise 5 or more tablets, 10 or more tablets, 15 or more tablets, 20 or more tablets, 25 or more tablets, 30 or more tablets, or 35 or more tablets, and 80 or less tablets, 70 or less tablets, 60 or less tablets, 50 or less tablets, 40 or less tablets, 30 or less tablets, 20 or less tablets, or 10 or less tablets, but is not limited to them, and may be appropriately adjusted depending on the tablet size.

The colonic purgative of the present disclosure may further improve medication convenience by taking it through the packaging bag as described above.

In addition, the present disclosure may relate to a pharmaceutical composition for the colonic purgative. On the day before the colonoscopy, 160 tablets of the colonic purgative tablet of claim 1 and 425 ml of water are taken, then 850 ml of water is taken in two additional doses of 425 ml each, and on the day of the colonoscopy, colonic purgative tablets and water are repeatedly taken in the same manner as above, and the total dose of the colonic purgative is 38 to 55 g. Each dosing time zone the day before and on the day of the test may be selected in a known manner depending on the time of performing the test.

In addition, the present disclosure may relate to a pharmaceutical composition for colonic purgative. On the day of the colonoscopy, 160 tablets of the colonic purgative tablet and 425 ml of water are taken, then 850 ml of water is taken in two additional doses of 425 ml each, and the total dose of the colonic purgative is 38 to 55 g. The time zone for each of the two doses on the day of the colonoscopy may be selected in a known manner depending on the time of performing the colonoscopy.

### MODE(S) FOR CARRYING OUT THE INVENTION

Hereinafter, the present disclosure will be described in detail with reference to examples in order to specifically explain the disclosure of the present disclosure as described above and the intended actions and effects of the present disclosure. However, Examples may be modified into various other forms, and the scope of the present specification is not to be construed as being limited to these Examples only. It is emphasized that the Examples represent the present disclosure and are provided to explain the present disclosure in more detail to those skilled in the art.

### Example 1

### Preparation of Uncoated Tablets

Anhydrous sodium sulfate, potassium sulfate, anhydrous magnesium sulfate, and simethicone were mixed in a high-speed mixer, and then polyethylene glycol (PEG 6000) was added and mixed. Thereafter, the mixed composition was compressed into tablets using a rotary tablet press to prepare uncoated tablets. At this time, the weight of each ingredient comprised in one uncoated tablet was as shown in Table 1 below.

### Formation of Coating Layer I

Sodium alginate was added to purified water and completely dissolved to prepare coating solution I. Subsequently, the uncoated tablet and coating solution I were placed in a film coating machine and coated under the conditions of an exhaust temperature of 40 to 60° C., a coating pan of 2 to 10 rpm, and a film coating solution pump of 20 to 80 rpm. The weight of sodium alginate comprised in one tablet formed through this was as shown in Table 1 below.

### Formation of Coating Layer II

A polyvinyl alcohol-polyethylene glycol graft copolymer was added to purified water and completely dissolved to prepare coating solution II. Subsequently, the sodium alginate coated tablet and coating solution II were placed in a film coating machine and coated under the conditions of an exhaust temperature of 40 to 60° C., a coating pan of 2 to 10 rpm, and a film coating solution pump of 20 to 80 rpm to prepare tablets. The weight of the polyvinyl alcohol-polyethylene glycol graft copolymer comprised in one tablet formed through this was shown in Table 1 below.

### Dosage Method

It may be taken in one of the following divided dosing or non-divided dosing.

### i) Divided Dosing

The evening before the colonoscopy, 40 tablets/1 pocket*4 pockets of this medicine and 425 mL of water were taken, and then an additional 850 mL of water (425 mL, twice) was taken. On the morning of the test, repeat the same dosage as the day before the test.

### ii) Non-Divided Dosing

40 tablets/1 pocket*4 pockets of this medicine and 425 mL of water were taken, and then an additional 850 mL of water (425 mL, twice) was taken. It was repeatedly taken on the day of the colonoscopy.

### Example 2

Tablets were prepared in the same manner as Example 1, except that the tablet composition and weight were as shown in Table 1 below.

Meanwhile, the tablet was taken in the same manner as Example 1, except that 14 tablets were taken instead of 40 tablets/1 packet*4 packets.

### Comparative Example 1

Tablets were prepared in the same manner as Example 1, except that the coating layer I was not formed. The dosage method was also the same as Example 1.

### Comparative Example 2

Tablets were prepared in the same manner as Example 1, except that sodium alginate was introduced into the uncoated tablets. The dosage method was also the same as Example 1.

### Comparative Example 3

Tablets were prepared in the same manner as Example 1, except that polaprezinc was introduced instead of sodium alginate as coating layer I. The dosage method was also the same as Example 1.

### Comparative Example 4

Tablets were prepared in the same manner as Example 2, except that the coating layer I was not introduced. The dosage method was also the same as Example 2.

TABLE 1

| | | | | | | Comparative | |
|---|---|---|---|---|---|---|---|
| | | Example 1 | | Example 2 | | Example 1 | |
| Classification | | Components | Doses | Components | Doses | Components | Doses |
| Tablet | Uncoated | Anhydrous | 98.44 | Anhydrous | 1125.00 | Anhydrous | 98.44 |
| composition | tablet | sodium | mg | sodium | mg | sodium | mg |
| | | sulfate | | sulfate | | sulfate | |
| | | Potassium | 17.59 | Potassium | 201.07 | Potassium | 17.59 |
| | | carbonate | mg | carbonate | mg | carbonate | mg |
| | | Anhydrous | 9.00 | Anhydrous | 102.86 | Anhydrous | 9.00 |
| | | magnesium | mg | magnesium | mg | magnesium | mg |
| | | sulfate | | sulfate | | sulfate | |
| | | Simethicone | 1.00 | Simethicone | 11.43 | Simethicone | 1.00 |
| | | | mg | | mg | | mg |
| | | Polyethylene | 15.42 | Copovidone | 29.64 | Polyethylene | 15.42 |
| | | glycol | mg | | mg | glycol | mg |
| | | — | — | — | — | — | — |
| | Coating | Sodium | 6.25 | Sodium | 71.45 | — | — |
| | layer I | alginate | mg | alginate | mg | | |
| | Coating | Polyvinyl | 2.30 | Polyvinyl | 30.00 | Polyvinyl | 2.30 |
| | layer II | alcohol- | mg | alcohol- | mg | alcohol- | mg |
| | | polyethylene | | polyethylene | | polyethylene | |
| | | glycol graft | | glycol graft | | glycol graft | |
| | | copolymer | | copolymer | | copolymer | |

| | | | |
|---|---|---|---|
| Tablet shape and size | Round (5 mm) | Rectangular (17 × 8 mm) | Round (5 mm) |
| 1 tablet weight | 150 mg | 1571.43 mg | 143.75 mg |

| | | Comparative | | Comparative | | Comparative | |
|---|---|---|---|---|---|---|---|
| | | Example 2 | | Example 3 | | Example 4 | |
| Classification | | Components | Doses | Components | Doses | Components | Doses |
| Tablet | Uncoated | Anhydrous | 98.44 | Anhydrous | 98.44 | Anhydrous | 1125.00 |
| composition | tablet | sodium | mg | sodium | mg | sodium | mg |
| | | sulfate | | sulfate | | sulfate | |
| | | Potassium | 17.59 | Potassium | 17.59 | Potassium | 201.07 |
| | | carbonate | mg | carbonate | mg | carbonate | mg |
| | | Anhydrous | 9.00 | Anhydrous | 9.00 | Anhydrous | 102.86 |
| | | magnesium | mg | magnesium | mg | magnesium | mg |
| | | sulfate | | sulfate | | sulfate | |
| | | Simethicone | 1.00 | Simethicone | 1.00 | Simethicone | 11.43 |
| | | | mg | | mg | | mg |
| | | Polyethylene | 15.42 | Copovidone | 15.42 | Polyethylene | 29.64 |
| | | glycol | mg | | mg | glycol | mg |
| | | Sodium | 6.25 | — | — | — | — |
| | | alginate | mg | | | | |
| | Coating | — | — | Polaprezinc | 0.47 | — | — |
| | layer I | | | | mg | | |
| | Coating | Polyvinyl | 2.30 | Polyvinyl | 2.30 | Polyvinyl | 30.00 |
| | layer II | alcohol- | mg | alcohol- | mg | alcohol- | mg |
| | | polyethylene | | polyethylene | | polyethylene | |
| | | glycol graft | | glycol graft | | glycol graft | |
| | | copolymer | | copolymer | | copolymer | |

| | | | |
|---|---|---|---|
| Tablet shape and size | Round (5 mm) | Round (5 mm) | Rectangular (17 × 8 mm) |
| 1 tablet weight | 150 mg | 144.22 mg | 1500.00 mg |

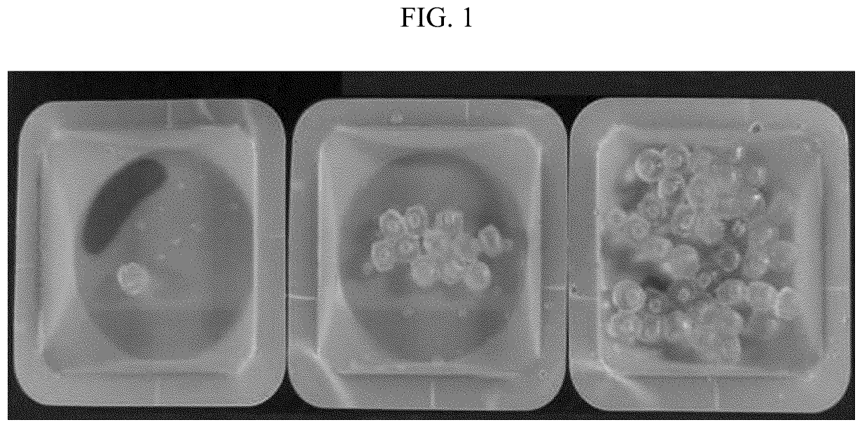

The tablets of Example 1 were grouped into 1 tablet, 11 tablets, and 40 tablets, and each was left in a 0.1 N HCl solution (pH about 1.2) at 25 to 30° C. for 30 minutes to check whether gelation occurred. As a result, it was confirmed that the coating layer comprising sodium alginate was gelled, as shown in FIG. 1.

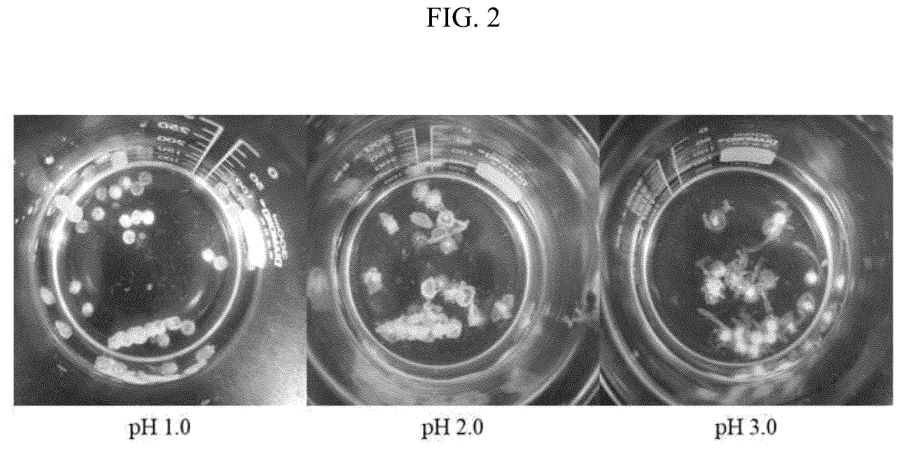

Meanwhile, the tablet of Example 1 was left in an HCl solution at pH 1.0, pH 2.0, and pH 3.0 at 25 to 30° C. for 30 minutes, and then the gelation pattern according to pH was confirmed. As a result, as shown in FIG. 2, it was confirmed that gelation was maintained under conditions of pH 1.0, 2.0, and 3.0. Through this, it can be expected that the coating layer comprising sodium alginate reacts with gastric acid to form a gel which is a physical defense factor, and even when the pH in the stomach rises slightly when ingested with a large amount of water, gelation is maintained and has an excellent gastric mucosal protection effect.

### Evaluation Example 2. Dissolution Pattern

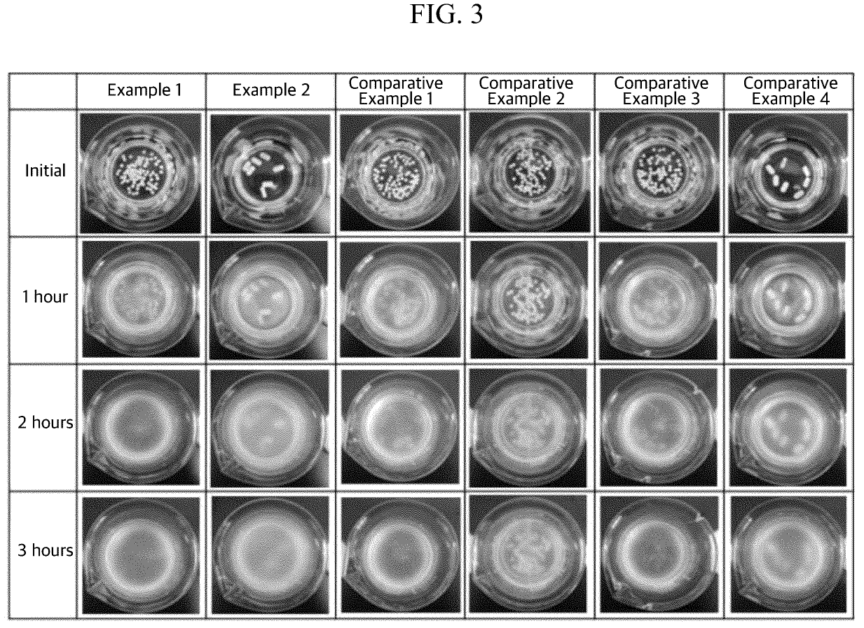

The tablets of Examples and Comparative Examples were placed in 410 mL of purified water in a beaker at about 25 to 30° C., and the beaker was shaken for about 1 hour. In Examples 1 and 2, sodium alginate was comprised in the uncoated tablet by introducing a coating layer comprising sodium alginate, in Comparative Examples 1, 2, and 4 in which no sodium alginate was introduced, the initial dissolution rate was faster compared to the size of the tablet, and the initial dissolution rate was similar to Comparative Example 3 coated with polaprezinc. Through this, in the case of the Examples, it can be expected that sodium alginate not only gels in the stomach, but also dissolves quickly compared to the tablet size, resulting in an excellent gastric mucosa protective effect (FIG. 3).

### Evaluation Example 3. Osmotic Pressure

As a result of preparing tablets of Examples and Comparative Examples according to the osmotic pressure measurement method among the general test methods of the Korean Pharmacopoeia and measuring the osmotic pressure of the tablet using Osmamat 3000 (manufacturer: Gonolec) as an analysis equipment, it was confirmed that there was no significant difference in osmotic pressure between the Examples and Comparative Examples as shown in Table 2 below. Through this, it can be expected that even when a gel-forming coating layer is introduced, the intestinal cleansing effect due to osmotic pressure or imbalance due to electrolytes would be low.

TABLE 2

| | Osmotic pressure |
|---|---|
| Classification | (mOsmol/kg) |
| Example 1 | 249 |
| Example 2 | 247 |
| Comparative | 241 |
| Example 1 | |
| Comparative | 246 |
| Example 2 | |
| Comparative | 242 |
| Example 3 | |
| Example 4 | 240 |

### Evaluation Example 4. Anhydrous Sodium Sulfate Elution Pattern

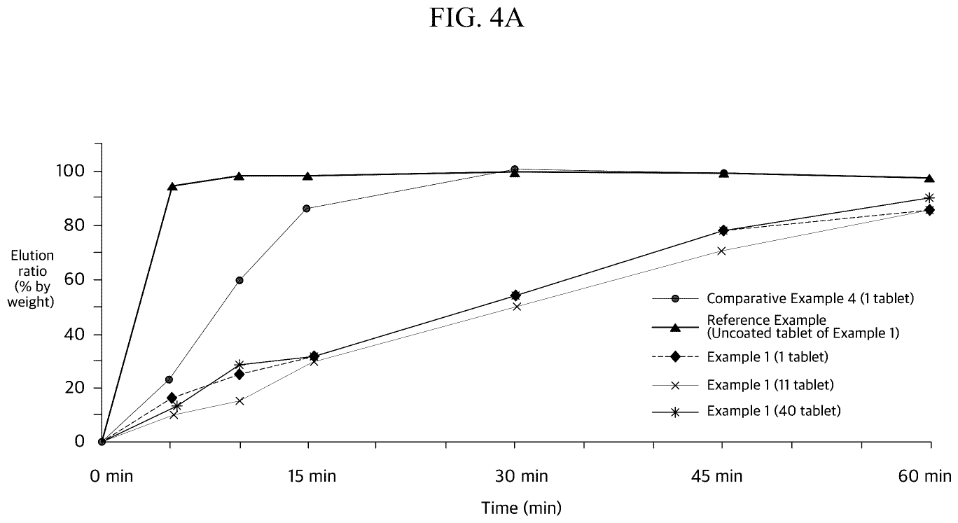

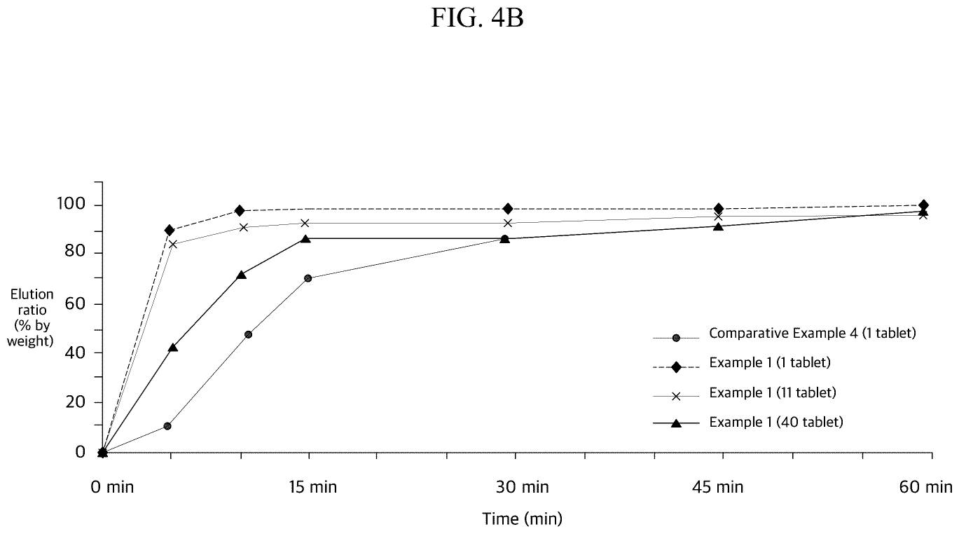

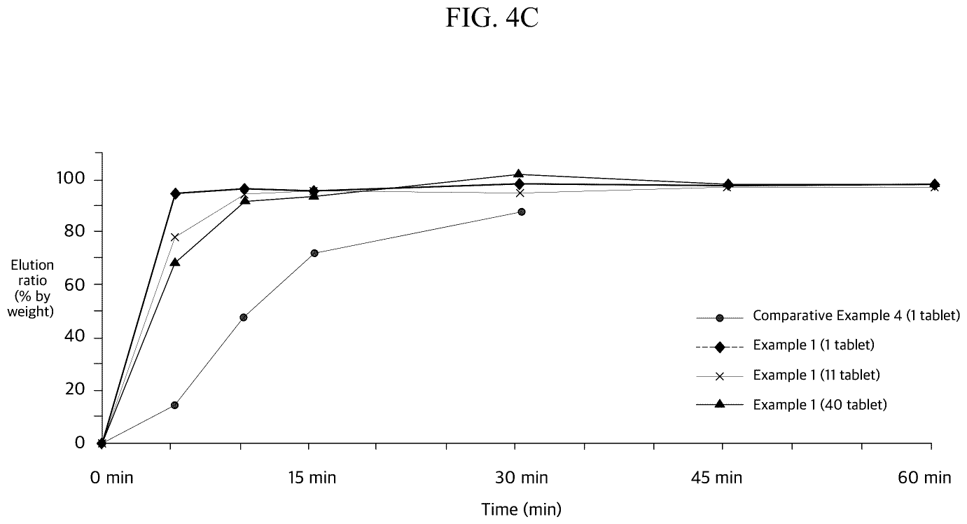

The elution pattern of anhydrous sodium sulfate of the tablets of Example 1 and Comparative Example 1 was evaluated according to the elution test method 2. Specifically, 900 ml of each test solution with pH 1.2 (0.1 N HCl), pH 4.0, and pH 6.8 was prepared, and the test solution was placed together with the tablet in a elution test device (paddle method), and then the elution pattern of anhydrous sodium sulfate over time was checked (under a temperature of 37±2° C.) while stirring at a speed of about 50 rpm. As a result, in Example 1, it was confirmed that the anhydrous sodium sulfate was eluted at less than 20% within 5 minutes at pH 1.2, more than 40% within 5 minutes at pH 4.0, and more than 60% within 5 minutes at pH 6.8, and while in Comparative Example 4, anhydrous sodium sulfate was eluted at more than 20% by weight within 5 minutes at pH 1.2, less than 10% by weight within 5 minutes at pH 4.0, and less than or equal to 15% by weight within 5 minutes at pH 6.8. Through this, it can be expected that the tablet of Example 1 will have an excellent intestine cleansing effect due to the high elution rate of osmotic substances in vivo (FIGS. 4(a) to (c)).

### Evaluation Example 5. Clinical Trial Results

### (1) Indications

Bowel cleansing before colonoscopy

### (2) Methodology

This clinical trial was designed and conducted as a prospective, randomization, single-blind, 3-treatment arm, parallel treatment group, active-controlled, and a multi-center study.

For clinical trial subjects who meet the selection criteria through the screening visit (V1), the investigational drug (test group 1 (Example 1 divided dosing), the test group 2 (Example 1 non-divided dosing), and the control group (Comparative Example 4 divided doses)) through the randomization visit (V2) dosing) were assigned, after taking the medication according to the individual usage (non-divided dosing—1 day/divided dosing—2 days), a visit for test (V3) was performed.

### (3) Usage and Dose

### <Test Drug (Example 1)>

Dose [standard for performing one colonoscopy]: 8 packets (40 tablets per packet)
Usage: 2 times (the day before and the day of the colonoscopy) or 1 time (the day of the colonoscopy), oral administration
Test group 1 (Example 1_Divided dosing): After taking 4 pockets of this medicine and 425 mL of water, an additional 850 ml of water (425 mL twice) was taken. The above process was repeated the day before and on the day of the test.
Test group 2 (Example 1_Non-split dosing): 4 pockets of this medicine and 425 mL of water were taken, and then an additional 850 mL of water (425 mL, twice) was taken. The above process was repeated 4 to 5 hours before the test.

### <Control Drug (Comparative Example 4)>

Dose [standard for performing one colonoscopy]: 28 tablets
Usage: 2 times (the day before and the day of the colonoscopy), oral administration
14 tablets of this medicine and 425 mL of water were taken, and then an additional 850 mL of water (425 mL, twice) was taken. The above process was repeated the day before and on the day of the test.

### (4) Treatment Group Assignment

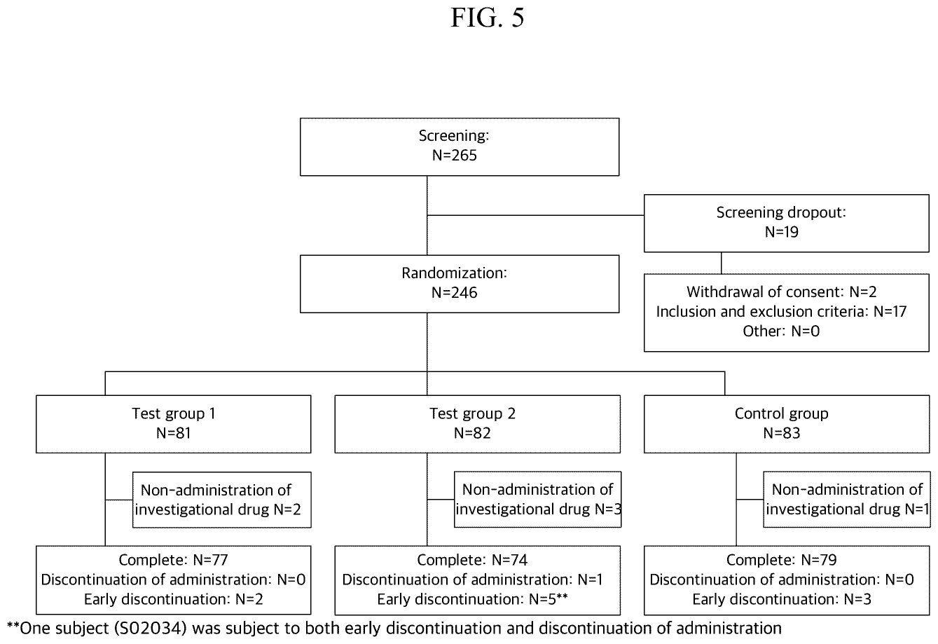

At the time of randomization, subjects were administered investigational drugs according to the designated treatment group. The number of subjects by treatment group was as shown in FIG. 5.

### (5) Statistical Analysis Group (Statistical Method)

### 1) Safety Analysis Group (Safety Set)

This refers to the population of subjects who, after being randomly assigned to this clinical trial, received at least one dose of the investigational drug and underwent safety-related follow-up.

### 2) FA Analysis Group (Full Analysis Set; FAS)

This refers to a population of subjects who received at least one administration of the investigational drug after being randomly assigned to this clinical trial, and for whom effectiveness evaluation was measured at least once after administration. In cases where rectal discharge is not clean despite taking the investigational drug (colon cleansing agent) or an adverse reaction occurred due to taking the investigational drug and a colonoscopy was not performed, when evaluating effectiveness variables related to colon cleanliness, the analysis was categorized as failure ‘unsuccessful cleansing’ according to the Harefield cleansing scale. In addition, even though a colonoscopy was performed after taking the investigational drug (colon cleansing agent), in the case of a segment in which colonoscopy could not be completed due to poor colon cleanliness, the segment is evaluated as 0 and when evaluating effectiveness variables, the analysis was categorized as failure ‘unsuccessful cleansing’ according to the Harefield cleansing scale.

### 3) PP1 Analysis Group (Principal Efficacy Analysis Set)

This refers to a population of subjects who received investigational drugs after randomization and underwent effectiveness evaluation at least once, without any serious protocol violations (violation of selection/exclusion criteria, violation of medication compliance, etc.). The medication compliance was set at 80%. Among the selection/exclusion criteria, BMI and violations of pregnancy test-related matters that were judged not to confound the effectiveness evaluation were not excluded from the evaluation.

### 4) PP2 Analysis Group

Among the PP1 group, this means the population of subjects who completed the clinical trial without any major violations.

### Statistical Analysis

The effectiveness evaluation targets the FAS, PP1 and PP2 groups, and the safety evaluation targets the safety group.

The principal effectiveness analysis group (principal effectiveness analysis set) is the PP1 group.

Missing values are not replaced with other values.

In the case of continuous data as descriptive statistics, the basic principles are mean, standard deviation, median, minimum and maximum values, and in the case of categorical data, frequency and ratio are the basic principles, and more detailed data are presented when necessary.

All statistical analyzes except non-inferiority evaluation are based on two-sided tests at a significance level of 0.05.

Gatekeeping method is applied to control the significance level to 5% or less in primary effectiveness evaluation. When test group 1 is confirmed to be non-inferior to the control group in hypothesis 1, hypothesis 2 is assayed without additional adjustment of the significance level.

### (6) Consideration and Overall Conclusion

### 1) Foam Removal Capacity

There was no statistically significant difference in the foam score evaluated using colonoscopy along with cleanliness, not only in the total score but also in the scores for each compartment regardless of whether the scores between test groups 1 and 2 and the control group were assessed by the evaluator (Effectiveness Evaluation Committee or colonoscopist).

Since it has already been confirmed that simethicone comprised in the control drug removes foam in the intestinal tract, it was judged that test groups 1 and 2 could also help secure visibility during colonoscopy by removing foam in the intestinal tract.

### 2) Subjective Dosage Satisfaction

There was no significant difference between treatment groups in subjective dosage satisfaction as assessed by the recipient. Therefore, it was determined that test groups 1 and 2 did not impede subjective dosage satisfaction the tablets compared to taking the control group.

### 3) Colonoscopy Quality Evaluation

Since the quality of colonoscopy in all treatment groups achieved the previously recommended level, the quality of colonoscopy performed in this clinical trial was judged to be very good.

### 4) Incidence Rate of Acute Injury to Gastric Mucosa Due to Drugs

Lower results were observed in the test group compared to the control group, but the incidence rate of acute injury to the gastric mucosa caused by the drug was not statistically significant between the treatment groups. However, in the proportion of subjects with a very high relationship with the drug (acute injury score of 8 or more), lower results were observed in test group 1 compared to the control group, and this was statistically significant.

In previous studies on the evaluation of acute gastric mucosal injury, the final determination of causality with the drug was made by an independent evaluator after scoring according to the evaluation criteria, whereas in this trial, only the scores according to the evaluation criteria were evaluated. In addition, in this trial, considering the fact that the degree of gastric injury at baseline cannot be confirmed because gastroscopy was not performed before administration of the investigational drug and the bias caused by the evaluator's subjective evaluation, the proportion of subjects with a highly drug-related acute injury score of 8 or more is considered a variable with greater clinical significance for comparing differences between treatment groups. As a result, it is believed that sodium alginate (alginic acid), which is an ingredient in the test drug, alleviated acute injury to the gastric mucosa.

### 5) Safety

### \textcircled{1} Adverse Events

No deaths were reported, and only one serious adverse event was reported that was not causally related to the investigational drug. Only one adverse event that resulted in dropout from the clinical trial was reported as an adverse drug reaction.

Considering the occurrence status of adverse events comprising the status of occurrence of serious adverse events and adverse events leading to dropout, adverse drug reactions, and adverse events ((1) abdominal inflation, (2) nausea, (3) vomiting, and (4) abdominal pain) scheduled to be collected in advance for tolerability evaluation, the safety profile of test groups 1 and 2 was similar to that of the control group, and the safety profile was judged to be good.

### \textcircled{2} Laboratory Test Values

It is well known that administration of the colon cleansing agent causes temporary fluctuations in hematology and blood chemistry tests. In particular, it is important to note that electrolyte fluctuations may cause the occurrence or worsening of arrhythmia or convulsion.

As a result of an inter-group comparison of normal/abnormal changes in hematology after administration (V3) compared to before administration (V2) for all hematology and blood chemistry test items, there were no significant differences between groups in all test items, and no clinically significant laboratory abnormalities were reported.

Among the hematology and blood chemistry test items, it was judged that the change pattern over time in some items was statistically significant, but most of them reflected physiological changes consistent with temporary decrease in body fluid and hemoconcentration due to diarrhea induction, which is the pharmacological action of colon cleansing agents. In addition, considering that most of the significant changes in the average change over time were reversible changes that normalized, and that the amount of change was also insignificant, the clinical significance of the change in the average test values of hematology and blood chemistry test items was judged to be small.

### \textcircled{3} Vital Signs, Physical Examination, and Electrocardiogram Findings

The average change in vital signs over time compared to before administration of the investigational drug showed no significant differences between treatment groups for all vital sign evaluation items, and changes (decreased systolic and diastolic blood pressure, increased pulse rate, weight loss, and tendency to decrease body mass index) within the group in vital signs on the day of colonoscopy over time after administration of the investigational drug were assumed to be findings consistent with body fluid reduction.

No subjects with clinically significant abnormalities on physical examination and electrocardiogram were reported.

### 6) Conclusion

When the above-mentioned matters were comprehensively evaluated, test groups 1 and 2 were satisfied with non-inferiority in terms of cleanliness using the Harefield scale compared to the control group. In addition, it helped secure the view of the colonoscopy by removing intestinal foam to a similar level as the comparison group, and was confirmed to be similar to Orafang® tab in terms of dosage ease and satisfaction, which are subjective evaluations felt by users of colon cleansing tablets.

As a result of analysis and evaluation of adverse events that occurred, systematic collection using questionnaires, evaluation of vital signs and physical examination findings, and laboratory test results such as hematology and blood chemistry tests, urine tests, and the like, it was confirmed that both test groups 1 and 2 showed no difference in safety from the control group, and were a colon cleansing agent with a good safety profile.

Considering all the above results, test groups 1 and 2 were judged to be good drugs in terms of safety while being non-inferior in terms of a colon cleansing effect for colonoscopy compared to the control group.

Specific results are shown in Tables 3 to 8 below.

TABLE 3

| | | Test | Test | Control |
|---|---|---|---|---|
| | | group 1 | group 2 | group |
| Classification | | (N = 78) | (N = 77) | (N = 78) |
| Taste | N | 78(100.00) | 76(100.00) | 78(100.00) |
| | A | 77(98.72) | 74(97.37) | 77(98.72) |
| | B | 1(1.28) | 2(2.63) | 1(1.28) |
| | C | 0(0.00) | 0(0.00) | 0(0.00) |
| | D | 0(0.00) | 0(0.00) | 0(0.00) |
| | P- | 1.0000(F) | 0.6176(F) | |
| | value* | | | |

*Chi-square test (C) or Fisher's exact test (F) (vs. control group)

Table 3 shows the proportion of each subject rated by categorizing them into four levels, A, B, C, and D, on the Harefield cleanliness scale evaluated by the evaluation committee.

In this specification, N refers to the number of subjects, A is a case where there is all segments with a score of 3 or 4, B is a case where there is one or more segments with a score of 2, C is a case where there is one or more segments with a score of 1, and D is a case where there is one or more segments with a score of 0. In this specification, segment refers to the rectum, sigmoid colon, descending colon, transverse colon, and ascending colon. Here, the HAREFIELD 5-level colon cleanliness scale score for each segment was scored according to the criteria below.
0 points: irremovable, heavy, hard stools
1 point: semi-solid, only partially removable stools
2 points: brown liquid removable semi-solid stools
3 points: clear liquid
4 points: empty and clean

TABLE 4

| | | | | Control |
|---|---|---|---|---|
| | | Test group 1 | Test group 2 | group |
| | Classification | (N = 78) | (N = 77) | (N = 78) |
| Harefield | N | 78 | 77 | 78 |
| cleanliness | A or B | 75(96.15) | 72(93.51) | 74(94.87) |
| scale | C or D | 3(3.85) | 5(6.49) | 4(5.13) |

| | | |
|---|---|---|
| A or B | [−6.33, ∞]* | [−8.67, ∞]* |
| 97.5% one-sided | | |
| confidence interval | | |
| vs control group | | |
| A or B | [−5.66, ∞] | [−7.00, ∞] |
| 97.5% one-sided | | |
| confidence interval | | |
| (CMH test) vs control group | | |

*Exact confidence limit (Score)

Table 4 reports the fraction of subjects rated as A or B or C or D on the Harefield cleanliness scale as assessed by the colonoscopy operator.

TABLE 5

| | | Test | Test | Control |
|---|---|---|---|---|
| | | group 1 | group 2 | group |
| Classification | | (N = 78) | (N = 77) | (N = 78) |
| Taste | N | 78 | 76 | 78 |
| | A | 62(79.49) | 56(73.68) | 61(78.21) |
| | B | 13(16.67) | 16(21.05) | 13(16.67) |
| | C | 2(2.56) | 3(3.95) | 3(3.85) |
| | D | 1(1.28) | 1(1.32) | 1(1.28) |
| | P-value* | 1.0000(F) | 0.9258(F) | |

*Chi-square test (C) or Fisher's exact test (F) (vs. control group)

Table 5 shows the proportion of each subject rated by categorizing them into four levels, A, B, C, and D, on the Harefield cleanliness scale evaluated by the colonoscopy operator.

TABLE 6

| | | Test | Test | Control |
|---|---|---|---|---|
| | | group 1 | group 2 | group |
| Classification | | (N = 78) | (N = 77) | (N = 78) |
| Taste | N | 78 | 76 | 78 |
| | No taste | 69(88.46) | 56(73.68) | 67(85.90) |
| | or good | | | |
| | taste | | | |
| | Not good | 8(10.26) | 18(23.68) | 10(12.82) |
| | but | | | |
| | bearable | | | |
| | Hard | 1(1.28) | 2(2.63) | 1(1.28) |
| | to bear | | | |
| | Inedible | 0(0.00) | 0(0.00) | 0(0.00) |
| | P-value* | 0.9007(F) | 0.1424(F) | |

*Chi-square test (C) or Fisher's exact test (F) (vs. control group)

Table 6 shows the proportion of each subject rated by categorizing them into four levels, ‘No taste or good taste’, ‘Not good but bearable’, ‘Hard to bear’, and ‘Inedible’, on the Harefield cleanliness scale evaluated after oral administration of the investigational drug.

TABLE 7

| | | | | Integration | |
|---|---|---|---|---|---|
| | | | | of test | |
| | | | | groups 1 | Control |
| | | Test group 1 | Test group 2 | and 2 | group |
| Classification | | (N = 78) | (N = 77) | (N = 155) | (N = 78) |
| Occurrence | N | 78 | 76 | 154 | 78 |
| of acute | Subjects | 63 | 65 | 128 | 73 |
| injury to | negative for | | | | |
| gastric | H. pylori | | | | |
| mucosa | Yes | 23(36.51) | 25(38.46) | 48(37.50) | 38(52.05) |
| | No | 40(36.49) | 40(61.54) | 80(62.50) | 35(47.95) |
| | P-value* | 0.0691(C) | 0.1095(C) | 0.0449(C) | |

*Chi-square test (C) or Fisher's exact test (F) (vs. control group)

Table 7 provides information on subjects who were negative for H. pylori (score: 3 or overall assessment NA) and who suffered acute injury to the gastric mucosa due to drugs assessed by the operator during gastroscopy after oral administration of the investigational drug.

TABLE 8

| | | | | Integration of | |
|---|---|---|---|---|---|
| | | | | test groups 1 | Control |
| | | Test group 1 | Test group 2 | and 2 | group |
| Classification | | (N = 78) | (N = 77) | (N = 155) | (N = 78) |
| 8 points or | N | 78 | 76 | 154 | 78 |
| more | Yes | 22(28.21) | 24(31.58) | 46(29.87) | 35(44.87) |
| | No | 56(71.79) | 52(68.42) | 108(70.13) | 43(55.13) |
| | P-value* | 0.0307(C) | 0.0898(C) | 0.0235(C) | |

*Chi-square test (C) or Fisher's exact test (F) (vs. control group)

Table 8 shows the results of the inter-group comparison of the proportion of subjects with an acute injury score of more than 8 among all subjects.

1. A colonic purgative tablet comprising:
an uncoated tablet comprising an osmotic substance, simethicone, and a water-soluble binder; and
a gel-forming coating layer comprising alginate as a main ingredient formed on the uncoated tablet,
wherein the osmotic substance comprises one or more selected from the group consisting of anhydrous sodium sulfate, potassium sulfate, anhydrous magnesium sulfate, sodium sulfate hydrate, magnesium sulfate hydrate, sodium picosulfate hydrate, light magnesium oxide, citric acid, polyethylene glycol, sodium chloride, potassium chloride, ascorbic acid, sodium ascorbate, sodium hydrogen carbonate, sodium hydrogen phosphate anhydride, sodium dihydrogen phosphate monohydrate, magnesium oxide, and potassium sulfate, and
the alginate is comprised in an amount of 50% by weight or more based on the total weight of the gel-forming coating layer.

2. The colonic purgative tablet of claim 1, wherein the osmotic substance comprises anhydrous sodium sulfate, potassium sulfate, and anhydrous magnesium sulfate.

3. The colonic purgative tablet of claim 2, comprising 60 to 90 parts by weight of anhydrous sodium sulfate, 5 to 25 parts by weight of potassium sulfate, and 1 to 15 parts by weight of anhydrous magnesium sulfate, based on 100 parts by weight of the osmotic substance.

4. The colonic purgative tablet of claim 1, comprising 0.1 to 2 parts by weight of simethicone and 0.5 to 20 parts by weight of a water-soluble binder based on 100 parts by weight of the osmotic substance.

5. The colonic purgative tablet of claim 1, wherein the water-soluble binder is one or more selected from the group consisting of copovidone, polyethylene glycol, and povidone.

6. The colonic purgative tablet of claim 1, wherein the alginate is one or more selected from the group consisting of sodium alginate, potassium alginate, and magnesium alginate.

7. The colonic purgative tablet of claim 1, wherein the gel-forming coating layer further comprises an adhesion improving agent.

8. The colonic purgative tablet of claim 7, wherein the adhesion improving agent is one or more selected from the group consisting of a polyvinyl alcohol-polyethylene glycol graft copolymer, hypromellose, hydroxypropyl cellulose, hydroxyethyl cellulose, polyester, gelatin, carboxymethyl cellulose, and povidone.

9. The colonic purgative tablet of claim 1, wherein the gel-forming coating layer further comprises a mucosa protective agent.

10. The colonic purgative tablet of claim 9, wherein the mucosa protective agent is one or more selected from the group consisting of an ecabet sodium hydrate, irsogladine maleate, and polaprezinc.

11. The colonic purgative tablet of claim 1, further comprising an adhesion-improving coating layer comprising an adhesion improving agent as a main ingredient on the gel-forming coating layer.

12. The colonic purgative tablet of claim 11, comprising 50% by weight or more of the adhesion improving agent based on the total weight of the adhesion-improving coating layer.

13. The colonic purgative tablet of claim 1, wherein when evaluating the elution pattern of the osmotic substance in a solution of pH 2 or lower according to the second elution test method, the osmotic substance is eluted at 30% by weight or less within 5 minutes.

14. The colonic purgative tablet of claim 1, wherein when evaluating the elution pattern of the osmotic substance in a solution of pH 3.5 to 5.5 according to the second elution test method, the osmotic substance is eluted at 30% by weight or more within 5 minutes.

15. The colonic purgative tablet of claim 1, wherein when evaluating the elution pattern of the osmotic substance in a solution of pH 6 to 8.5 according to the second elution test method, the osmotic substance is eluted at 40% by weight or more within 5 minutes.

16. A colonic purgative comprising 2 or more packaging bags comprising 2 or more tablets of the colonic purgative tablet of claim 1, and taking the colonic purgative tablet comprised in one packaging bag for a single time.

17. A pharmaceutical composition for colonic purgative, wherein on the day before the colonoscopy, 160 tablets of the colonic purgative tablet of claim 1 claim 1 and 425 ml of water are taken, then 850 ml of water is taken in two additional doses of 425 ml each, and on the day of the colonoscopy, colonic purgative tablets and water are repeatedly taken in the same manner as above, and the total dose of the colonic purgative is 38 to 55 g.

18. A pharmaceutical composition for colonic purgative, wherein on the day of the colonoscopy, 160 tablets of the colonic purgative tablet of claim 1 claim 1 and 425 ml of water are taken, then 850 ml of water is taken in two additional doses of 425 ml each, and the total dose of the colonic purgative is 38 to 55 g.
